(19) United States
(12) Patent Application Publication
(10) Pub. No.: US 2002/0169115 A1
(43) Pub. Date: Nov. 14, 2002

(54) Method of predicting resistance to hepatitis C infection

(75) Inventors: Mark R. Thursz, London (GB); Howard C. Thomas, London (GB)

Correspondence Address: Nixon & Vanderhye P.C., 8th Floor, 1100 N. Glebe Rd., Arlington, VA, 22201, US

(73) Assignee: Imperial College of Science Technology & Medicine

(21) Appl. No.: 09/750,774

(22) Filed: Jan. 2, 2001

Related U.S. Application Data
(63) Continuation of application No. 09/038,984, filed on Mar. 12, 1998, now abandoned.

(30) Foreign Application Priority Data
Jul. 12, 1996 (GB) 9614671.7
Jul. 14, 1997 (GB) PCT/GB97/01904

(51) Int. Cl. A61K 38/17; C12Q 1/70

(52) U.S. Cl. 514/12; 530/324; 435/5

(57) ABSTRACT

Method of identifying resistance to Hepatitis C infection wherein the presence of HLA-DRB1*1301-3, HLA-DRB1*1201, HLA-DRB1*1101 or HLA-DRB1*0701 are identified. Peptides capable of modifying immune response and methods of treating Hepatitis C are also disclosed.

The present invention relates to a novel method of predicting resistance or susceptibility to Hepatitis C, peptides useful in modifying immune response to Hepatitis C, vaccines against Hepatitis C and methods of treating Hepatitis C.

Assays to detect circulating antibodies to Hepatitis C virus (HCV) are still being refined. Results of epidemiological studies suggest that the carrier rate varies from about 0.2% in Northern Europe through 1 or 2% in the Mediterranean to 5% or more in the Far East. It is an important cause of chronic liver disease, cirrhosis and hepatocellular carcinoma (Souhami & Moxham in Textbook of Medicine 2nd edition 628-629 (1994)).

HCV is transmitted mainly by parenteral inoculation. Blood transfusion was one of the most important sources until the recent introduction of reliable blood tests for the detection of carriers. Acute hepatitis leads to chronic carriage of the virus in more than 50% of cases, but the degree of liver damage is often very mild, and it may be many years before significant chronic liver disease is seen. This means that liver function tests alone are very unreliable in determining whether or not recovery has occurred. Serological tests, or detection of the viral RNA in serum, are better indicators of the outcome (Souhami & Moxham in Textbook of Medicine 2nd edition 628-629 (1994)).

As an example, in the case of viral injection by Hepatitis B, recognition of foreign antigens by T lymphocytes is achieved through the presentation of antigenic peptides in the groove of MHC encoded HLA molecules. Such immunological responses are MHC restricted, meaning that foreign antigens are only recognised when presented by specific class I or class II molecules. In patients with acute Hepatitis B, class I restricted cycotoxic T lymphocytes (CTL) are present in the peripheral blood which recognise the nucleocapsid and envelope antigens of HBV (Bertolerti, A. et al, Proc. Natl. Acad. Sci, USA, 88(23): 10445-9, 1991). In addition there as a strong class II restricted proliferative response to the nucleocapsid antigens (Ferrari, C. et al, J. Clin. Invest, 88: 214-22, 1991). CD4⁺ T helper cell responses to the nucleocapsid and envelope antigens of the virus are required to support anti-HBe and anti-HBs antibody development (Milich, D. et al, Nature, 319: 547-9, 1967). in patients with chronic Hepatitis B virus (HBV) injection, CTL are not detectable and the proliferative response is absent or significantly reduced (Ferrari, C. et al, J. Immunol., 145(10): 3442-9, 1990, and Tsai, S. et al, J. Clin. Invest., 89: 87-96 1992).

Several studies have examined the role of MHC phenotype in the outcome of HBV infection without reaching a firm conclusion (Van-Hattum, J. et al, Hepatology, 7(1): 11-14, 1957; Forzani, B. et al, Hepatology, 4: 1107-10, 1984; Lepage, V. et al, Tissue Antigens, 18: 105-7. 1981; and Kaslow, R. and Shaw, S., Epidemiol. Rev. 3: 90-114, 1981). However, the majority of these studies have been of a size that could only detect a very strong association between MHC phenotype and disease state. Furthermore the sensitivity of these studies has been compromised by using serologically defined MHC class II typing methods which may assign a large number of alleles to the same serological specificity. DRB1*1302 is associated with a reduced risk of cerebral malaria in Gambian children, which may relate to the high frequency of the DRB1*1302-DRB3*0301-DQA1*0102-DQB1*0501 and DRB1*1302-DRB3*0301-DQA1*0102-DQB1*0604 haplotypes in this population (Lepage, V. et al, supra). The combined haplotype frequency in North Europeans is 4.4% compared with 16.4% in The Gambia (Hill, A. et al, Nature, 352(6336), 595-600, 1991). Malaria and HBV are both important causes of premature mortality in West Africa. However, whereas severe malaria has a high mortality rate in children under 5, HBV related mortalities are common during working life. If recovery from HBV infection were also linked to DRB1*1302, in addition to malaria, HBV would confer a selective advantage on individuals carrying the DRB1*302 haplotypes in The Gambia.

A recent study has shown a protective effect against human papilloma virus (HPV) related cervical carcinoma associated with DRB1*1302 and DRB1*1301 (Apple, R. J. et al, Nature Genetics, 6(2): 157-162, 1994).

DRw6, which is the serological supertype of DRB1*1302 and DRB1*1301, was identified in two previous studies as potentially protective against persistent HBV infection. Van Hattum et al found DRw6 nearly twice as frequently in North European patients who cleared HBV than in those who failed to eliminate the virus (Van-Hattum, J. et al, supra), In a study of factors influencing the response of chronic HBV infection to interferon therapy, we found that DRw6 is associated with a favourable response (Scully, L. et al, Hepatology, 12: 1111-17, 1990). However, both these studies were too small to reach statistical significance.

A method of identifying resistance to Hepatitis B has been described in International Patent Application No. PCT/GB95/02067, publication no. WO 96/07915. However, the viruses responsible for Hepatitis B and Hepatitis C infections are very different.

The virus responsible for Hepatitis C is much less well understood than the Hepatitis B virus. However, recent studies have identified several points of major distinction between the two viruses which seem to share only their specificity for causing disease in the liver. Hepatitis C is an RNA virus. It belongs to the Flavivirus family and is quite common throughout the world. Until relatively recently, the existence of the virus was unknown and individuals with illnesses displaying characteristics of viral hepatitis but which could not be identified as either Hepatitis A or Hepatitis B viral infections, were referred to as “non-A, non-B” infections. However, the Hepatitis B virus (HBV) is a DNA virus. HBV has an outer and inner protein coat, with double-stranded DNA and a DNA polymerase in the nucleocapsid. The HBV coat proteins are also quite conserved in contrast to the coat proteins of HCV which are variable. In patients infected with HBV, the virus is present in high quantities and the disease is self-limiting in 95% of cases. Whereas, patients infected with HCV have a persistent infection in 80% of cases and the virus is present in only low quantities. The Hepatitis C virus is therefore quite distinct from the virus responsible for Hepatitis B.

Following exposure to Hepatitis C virus (HCV) it is estimated that a patient will spontaneously eliminate the virus in 20-25% of cases and will develop a persistent infection in 75-80% of cases. No host or viral variables have yet been identified which determine this outcome.

In patients with persistent HCV infection approximately 20% will develop significant liver injury which will progress to cirrhosis and/or hepatocellular carcinoma. A number of viral variables, such as titre, genotype and diversity of quasi-species, have been shown to influence this outcome. Recently a number of authors have shown. data which suggests that HLA-DR5 protects against this progressive liver disease.

Patients with HCV infection may be treated with interferon a but a sustained response to therapy will only be seen in 25% of cases. The same viral variables which determine liver injury also appear to affect the outcome of interferon therapy. No host genetic factors have been identified which determine the outcome of interferon therapy.

CD4⁺ T helper cell responses which are in part determined by the host MHC genotype appear to protect patients against HCV induced progressive liver injury and are better in patients who respond to interferon therapy.

It has now been found that the presence of certain HLA molecules confers resistance to infection by Hepatitis C.

Accordingly, the present invention provides a method of identifying resistance to Hepatitis C infection which comprises the step of identifying the presence of HLA-DRB1*1301-3 or HLA-DRB1*1201 or HLA-DRB1*1101. The present invention also provides a method of identifying susceptibility to Hepatitis C infection which comprises the step of identifying the presence of HLA-DRB1*0701. Suitably identification is carried of on a sample of blood. Thus, the invention provides a convenient method of predicting Hepatitis C resistance in any given individual and hence also allows for predictions to be made concerning the outcome of Hepatitis C infection in individual patients.

In one embodiment the method of the present invention further comprises the step of identifying the presence of HLA-DRB1*1301-3 and/or HLA-DRB1*1201 and/or HLA-DRB1*1101. Again, this is suitably carried out by analysing a blood sample.

Furthermore, in view of the recognition that HLA-DRB1*1301-3 and/or HLA-DRB1*1201 and/or HLA-DRB1*1101 is/are associated with resistance to Hepatitis C. It is possible to modify the immune response of an individual by means so peptides which bind to one or both of those HLA molecules. In another aspect therefore, the present invention also provides one or more peptides capable of binding to HLA-DRB1*1301-3 and/or HLA-DRB1*1201 and/or HLA-DRB1*1101. Such peptides can be used to modify the ability of HLA-DRB1*1301-3 and/or HLA-DRB1*1201 and/or HLA-DRB1*1101 to elicit an immune response. Such an approach would be useful in immunization against HCV infection, and in DRB1*1301-3, DRB1*1201 and DRB1*1101 individuals, who had become persistently infected, would facilitate recovery (therapeutic immunisation). In a preferred embodiment, binding of the one or more modified peptides will result in a reduced immune response. This is particularly advantageous in treating conditions such as fulminant Hepatitis C.

More generally the present invention provides a peptide capable of modifying the ability of an HLA to elicit an immune response in response to a Hepatitis C antigen.

Preferably the peptide is one derived from a Hepatitis C antigen. For example, the peptide can consist only of a particular region known to bind to a particular HLA molecule. Alternatively, a synthetic peptide could be constructed consisting of a binding region and other, non-binding regions.

The peptides can be administered in the form of a pharmaceutical formulation, eg. as an intravenous formulation. Thus, in another aspect the invention provides a pharmaceutical formulation comprising one or more peptides capable of binding to HLA-DRB1*1101-3 and/or HLA-DRB1*1201, and/or HLA-DRB1*1101 together with one or more pharmaceutically acceptable carriers and/or excipients.

In addition, such peptides can be used in the production of a vaccine against Hepatitis C. Accordingly, the invention also provides a vaccine against Hepatitis C comprising one or more peptides capable of binding to HLA-DRB1*1301-3 and/or HLA-DRB1*1201 and/or HLA-DRB1*1101.

The peptides of the invention also provide other methods of treating Hepatitis C. For instance, antigen specific lymphocytes can be generated in vitro using a peptide of the invention. These can then be administered to a patient suffering from Hepatitis C. The present invention therefore also provides a composition comprising lymphocytes wherein the lymphocytes have been exposed to one or more peptides of the invention. A method of treating Hepatitis C is also provided which comprises the step of administering such a composition to a subject.

In general the lymphocytes will either be from the subject being treated or from another with a similar HLA type.

The invention will now be described by means of the following example which should not be construed as limiting the scope of the invention in any way.

### EXAMPLE 1

### Immungenetics of Hepatitis C Infection

All patients in the study which forms the basis for the following example were recruited to the study via the UK Lookback project in which patients who received HCV-contaminated blood prior to the introduction of screening were identified by the Blood Transfusion Service.

Acute (self-limiting) infection is defined in those patients who were tested positive or indeterminant with the Chiron HCV RIBA test and were tested negative on the Roche Amplicor HCV-PCR test. Persistent HCV infection was defined in those patients who were positive in both tests.

TABLE 1

| | Acute (Self | Chronic | | | |
|---|---|---|---|---|---|
| MHC | limiting) | (Persistent) | Odds ratio | | |
| Alleles | N = 17 | N = 58 | (95% Cl) | | P |
| 0101/4 | 2 | 7 | 0.97 | (0.09-5.88) | 0.99 |
| 0102 | 0 | 4 | 0 | (0-5.26) | 0.57 |
| 0301/4 | 5 | 22 | 0.68 | (0.17-2.45) | 0.51 |
| 0401-22 | 5 | 14 | 1.31 | (0.31-4.92) | 0.66 |
| 0701 | 1 | 16 | 0.16 | (0.01-1.26) | 0.06 |
| 0801-11 | 0 | 1 | 0 | (0-133.0) | 0.99 |
| 1101/4/6 | 2 | 7 | 0.97 | (0.09-5.88) | 0.99 |
| 1201 | 2 | 2 | 3.73 | (0.25-54.3) | 0.22 |
| 1301-3 | 7 | 5 | 6.75 | (1.48-31.5) | 0.002 |
| 1401/7 | 0 | 2 | 0 | (0-18.45) | 0.99 |
| 1501/3/4/5 | 4 | 14 | 1.19 | (0.24-4.68) | 0.75 |
| 1601 | 0 | 1 | 0 | (0—133.0) | 0.99 |

The data in Table 1 shows that MHC genotype influences the outcome of initial HCV infection by increasing or reducing susceptibility to persistent HCV infection. In particular it can be seen that DRB1*1301-3 and DRB1*1201 (an allele whose serological classification would be DR5) or HLA-DRB1*1103 reduce susceptibility to persistent HCV infection. On the other hand, DRB1*0701 increases susceptibility to persistent HCV infection.

It is therefore possible to use host MHC genotype to help predict the possibility of persistent HCV infection, to predict the possibility of significant liver injury and to predict the likely response to interferon therapy.

In this study we have used, as an example, the polymorphism of MHC class II at the DRB1 locus. It should also be possible to improve the prognostic accuracy using other polymorphisms in the MHC class I, II and III regions such as DQA1, DQB1, DPA1, TAP I & II, DM, A, B, and C.

1. A method of identifying resistance to Hepatitis C infection which comprises the step of identifying the presence of HLA-DRB1*1301-3.

2. A method of identifying resistance to Hepatitis C infection which comprises the step of identifying the presence of HLA-DRB1*1201.

3. A method of identifying resistance to Hepatitis C infection which comprises the step of identifying the presence of HLA-DRB1*1101.

4. A method of identifying susceptibility to Hepatitis C infection which comprises the step of identifying the presence of HLA-DRB1*0701.

5. A method as claimed in any one of claims 1 to 4 wherein identification is carried out on a blood sample.

6. A method of predicting the outcome of Hepatitis C infection in a patient which comprises identification of the patient's tissue type.

7. A peptide capable of binding to HLA-DRB1*1301-3 and/or HLA-DRB1*1201 and/or HLA-DRB1*1101.

8. A peptide as claimed in claim 7 capable of modifying the ability of HLA-DRB1*1301-3 and/or HLA-DRB1*1201 and/or HLA-DRB1*1101 to elicit an immune response.

9. A peptide capable of modifying the ability of an HLA to elicit an immune response in response to a Hepatitis C antigen.

10. A peptide as claimed in any one of claims 7 to 9 which is derived from a Hepatitis C antigen.

11. A pharmaceutical formulation comprising a peptide as defined in any one of claims 7 to 10, together with one or more pharmaceutically acceptable carriers and/or excipients.

12. A pharmaceutical formulation as claimed on claim 11 which is for intravenous administration.

13. A vaccine against Hepatitis C comprising one or more peptides as defined in any one of claims 7 to 10.

14. A composition comprising lymphocytes which have been treated with one or more peptides as defined in any one of claims 7 to 10.

15. A method of modifying the immune response of a subject to Hepatitis C which comprises the step of administering to the subject one or more peptides as defined in any one of claims 7 to 10.

16. A method for the treatment or prophylaxis of Hepatitis C which comprises the step of administering to a subject one or more of the peptides as defined in any one of claims 7 to 10.

17. A method for the treatment or prophylaxis of Hepatitis C which comprises the step of administering to a subject a pharmaceutical formulation as defined in claim 11 or claim 12.

18. A method for the treatment or prophylaxis of Hepatitis C which comprises the step of administering to a subject a vaccine as defined in claim 13.

19. A method for the treatment or prophylaxis of Hepatitis C which comprises the step of administering to a subject a composition as claimed in claim 14.
